(19) United States
(12) Patent Application Publication
(10) Pub. No.: US 2016/0278971 A1
(43) Pub. Date: Sep. 29, 2016

(54) STOMA PROTECTIVE DEVICE

(71) Applicant: Arlanders Taylor, JR., Elgin, IL (US)

(72) Inventor: Arlanders Taylor, JR., Elgin, IL (US)

(21) Appl. No.: 15/019,613

(22) Filed: Feb. 9, 2016

Related U.S. Application Data
(60) Provisional application No. 62/136,790, filed on Mar. 23, 2015.

(51) Int. Cl. A61F 5/445

(52) U.S. Cl. CPC A61F 5/445

(57) ABSTRACT

A stoma protective device is provided, comprising: a circular base ring having a top surface; a cylindrical wall extending from the top surface of the base ring and having a diameter less than a diameter of the base ring; and a hood extending from the cylindrical wall, the hood having a funnel shape with an opening through a top portion extending through at least a portion of a side portion. The base ring is configured to be secured between a flange secured to a patient's stoma and a ring secured to an ostomy pouch, whereby the hood protrudes into the interior of the ostomy pouch.

### RELATED APPLICATION DATA

The present application is related to and claims the benefit of commonly-owned and co-pending U.S. Application Ser. No. 62/136,790, entitled STOMA GUARD AND ASSOCIATED USE THEREOF, filed on Mar. 23, 2015, which application is incorporated herein by reference in its entirety.

### TECHNICAL FIELD

The present invention is related generally to post-surgical ostomy devices and, in particular, to a stoma protective device for use by a user who wears an ostomy bag.

### BACKGROUND ART

Every day, thousands of patients across the country undergo stoma surgical procedures resulting in a permanent or temporary ostomy. An ostomy is an artificial opening in the abdomen through which a portion of the patient's bowel is pulled, thus allowing solid or fluid waste matter to exit the body without passing through the rectum or urethra. This surgery is performed for a variety of reasons including abdominal obstruction, diseases such as cancer and ulcerative colitis, as well as because of trauma or congenital defects.

Ostomy procedures are broken into three major categories; urostomy, ileostomy and colostomy. A urostomy is a procedure where the ureters are detached from the bladder and joined to a small section of the small intestine to form a stoma. Urine is then excreted through this stoma and collected in an external urostomy pouch. Like a urostomy, an ileostomy involves bringing a small portion of the small intestine through an opening in the abdominal wall to form a stoma. Waste matter then exits this stoma and is collected in an external ileostomy pouch.

A similar procedure to the urostomy and ileostomy, a colostomy involves creating a stoma with a portion of the large intestine, as opposed to the small. Solid waste then passes through this stoma where it is collected in a colostomy pouch. Ostomy pouching systems may include a one-piece or two-piece system, each including a wafer (or face plate flange) and a collection pouch. The pouch (one-piece or two-piece) attaches to the abdomen by the flange and is fitted over and around the stoma to collect the diverted output, either stool or urine. This barrier is designed to protect the skin from the stoma output and to be as gentle on the skin as possible.

While stoma surgery can be a lifesaving and necessary procedure, adapting to life with an ostomy pouch can be difficult. Learning the proper care and maintenance for both a stoma and an ostomy pouch can be somewhat overwhelming during the days after surgery. In particular, many consumers who have undergone an ostomy procedure find that the process of preparing an ostomy flange for attachment to the stoma can be extremely challenging. Ostomy flanges are produced in two versions; pre-cut and cut-to-fit. Pre-cut flanges are flanges where the opening of the barrier is pre-cut according to standard sizes determined by the flange manufacturer.

Alternately, cut-to-fit flanges must be manually cut by the ostomy patient in accordance to the size of his or her stoma. Because the sizes available for pre-cut flanges are limited, many patients rely on cut-to-fit flanges in order to ensure that the flange is customized to meet their own, individual needs. Regardless of model, ostomy flanges and stomas can present a challenge to wearers. It is not uncommon for the stoma to be blocked by the waistband of pants or a belt of a dress or a car seat-belt could cause premature leaking out of the side of the flange or wafer.

As imagined, this can be both uncomfortably messy and embarrassing, particularly when there is an odor present. Besides the obvious discomfort, odor, and embarrassment associated with a leaking ostomy pouch, this type of leakage leads to one of the most common problems experienced by ostomy patients: peristomal skin complications. Peristomal skin complications can range from everything from rashes and burning to serious infections and breakdown of the skin around the stoma site. As such, making sure that the stoma is securely closed is of utmost concern for most ostomy patients.

### SUMMARY OF THE INVENTION

Embodiments of the present invention address the these common issues by providing a device configured to shield and protect a stoma from contacting and being blocked by a user's clothing, a seat-belt, or other external factors. The device is also convenient and easy to use and lightweight, yet durable in design, versatile in its applications, and designed for preventing premature leaks, backups, odor, embarrassment, and possible health problems that could otherwise occur.

### BRIEF DESCRIPTION OF THE DRAWINGS

FIG. 1A illustrates a front perspective view of an embodiment of a stoma protective device of the present invention;

FIG. 1B illustrates a rear perspective view of the stoma protective device of FIG. 1A;

FIG. 2A illustrates a top view of the stoma protective device of FIG. 1A;

FIG. 2B illustrates a cross-sectional view of the stoma protective device of FIG. 1A taken along section line BB in FIG. 2A;

FIG. 2C illustrates a cross-sectional view of the stoma protective device of FIG. 1A taken along section line C in FIG. 2A;

FIG. 3 illustrates a cut-away view of the stoma protective device of FIG. 1A secured between an ostomy pouch and a base; and

FIG. 4 illustrates the stoma protective device of FIG. 1A secured between an ostomy pouch, shown in cross-section, and a base.

### DETAILED DESCRIPTION OF THE PREFERRED EMBODIMENT

The described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention can be practiced without one or more of the specific details, or with other methods, components and so forth. In other instances, well-known structures, materials, or operations are not shown or described in detail to avoid obscuring aspects of the invention.

Non-limiting embodiments of the present disclosure are referred to generally in the figures and are intended to provide a hood or guard for use by a user who wears an ostomy bag. The hood or guard is configured to shield and protect a stoma from contacting, and being blocked by, a user's clothing, a seat-belt, or other external factors and thereby preventing premature leaks, backups, odor and embarrassment, and possible health problems that could otherwise occur. It should be understood that the embodiments may be used to guard/shield a variety of stomas, and should not be limited to any particular type of stoma described herein.

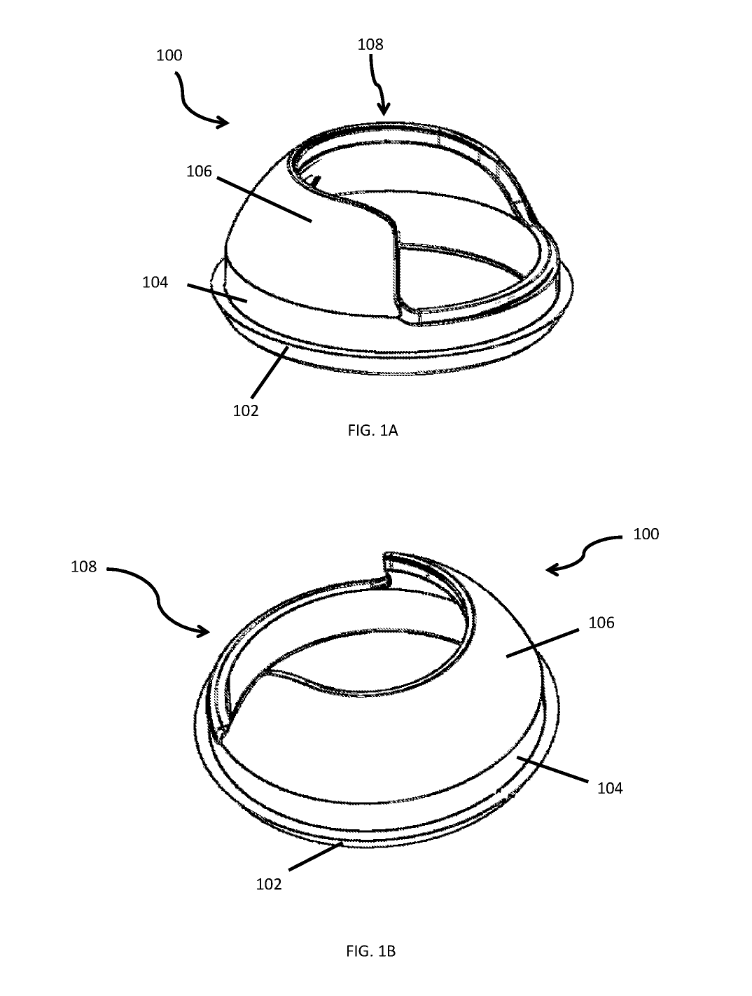

Referring to FIGS. 1A, 1B, an embodiment of a stoma protective device, referred to herein as a stoma guard 100 of the present invention is illustrated. The stoma guard 100 includes a circular base ring 102 and a cylindrical wall 104 positioned on the upper surface of the base ring 102. A hood 106 is positioned on the upper surface of the wall 104. The base ring 102 is preferably tapered outward from a first diameter at the bottom to a second, larger diameter at the top surface. The wall 104 extends upward from the top surface of the base ring 102 and has a diameter less than the diameter of the top surface. The bottom of the hood 106 has a diameter equal to the diameter of the wall 104 and is tapered inward as it extends above the wall 104 in a funnel-like shape. The hood 106 has an opening 108 formed in part of the top and part of the sidewall, as illustrated in FIGS. 1A and 1B.

The base ring 102, wall 104, and hood 106 is preferably formed, such as by molding, as a single integrated piece although other manufacturing processes may be used. The base ring 102, wall 104, and hood 106 may be formed from any appropriate biologically inert material that may be provided to the patient in sterile condition.

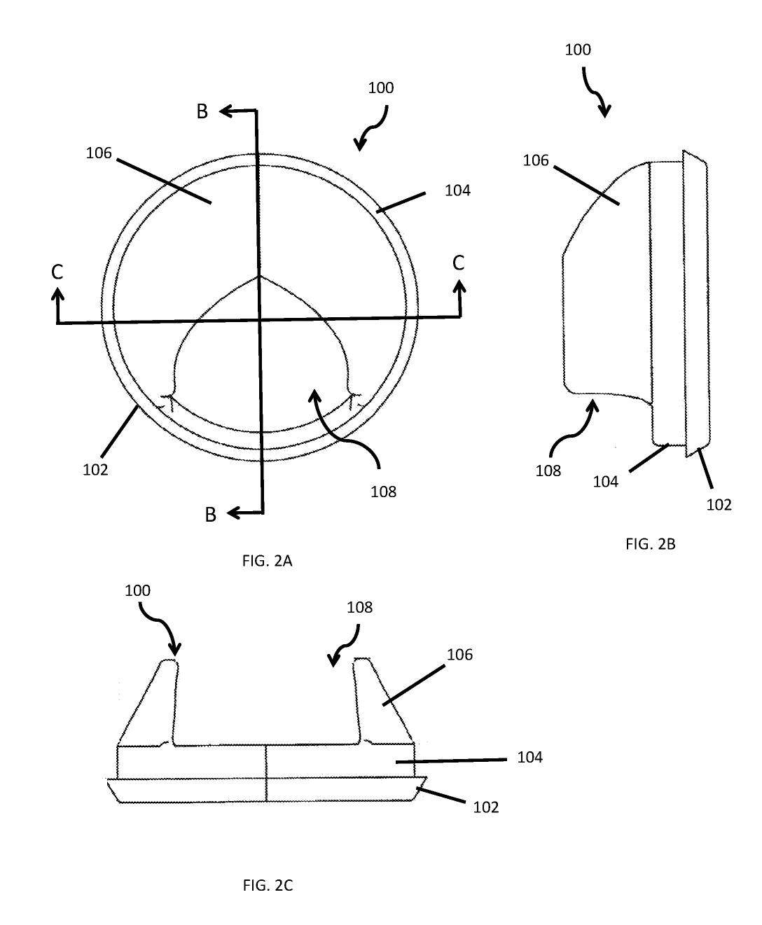

FIG. 2A, 2B, 2C illustrate top, front cross-section, and side cross section views, respectively, of the stoma guard 100. Viewed from above, as shown in FIG. 2A, the opening 108 is somewhat triangular in shape, although other shapes may be used as well. In one embodiment, the base ring 102 has diameter at the upper surface of about 0.4716 inches and a height of about 0.0035 inches. The cylindrical wall 104 and the bottom of the hood 106 have diameters of about 0.4356 inches. The height of the wall 104 above the base ring 102 is about 0.0503 inches and the height of the hood 106 above the wall 104 is about 0.013 inches. More generally, the base ring 102 may have a diameter at the upper surface from about 0.25 inches to about 0.5 inches and a height from about 0.002 inches to about 0.003 inches. The cylindrical wall 104 and the bottom of the hood 106 may have diameters from about 0.25 inches to about 0.5 inches. The height of the wall 104 above the base ring 102 may be from about 0.04 inches to about 0.06 inches and the height of the hood 106 above the wall 104 may be from about 0.01 inches to about 0.02 inches. However, it will be appreciated that the stoma guard 100 may manufactured to other dimensions to fit stomas of different sizes.

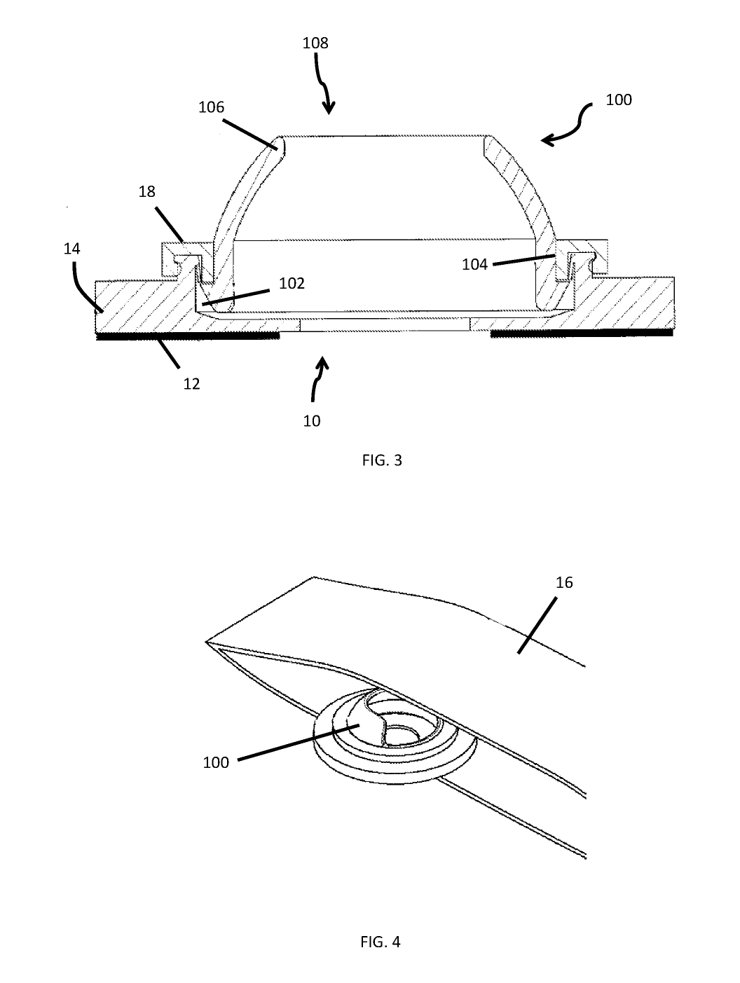

FIG. 3 illustrates a cross-sectional view of the stoma guard 100 in place over the stoma 10 of a patient. An adhesive 12 secures a flange 14 to the stoma 10. The hood 106 of the stoma guard 100 protrudes into the interior of the pouch 16 (FIG. 4) and is secured in place when the pouch 16 is secured to the flange 14 with a bag ring 18. Thus, the stoma guard 100 is configured to fully cover the egress tubing or stoma 10. In this manner, the stoma guard 100 keeps the stoma 10 and flange 14 securely encased until the user is ready to open it. A snap lock design ensures a tight fit to secure the stoma guard 100 between the flange 14 and bag ring 16 and prevent shifting as a result of the waistband or belt. When it is time to empty and clean the pouch 18, a user need only remove the pouch 18, attend to the ostomy pouch 18, and replace it when finished. Thus, the process of maintaining one's pouch hygienically would not be any different than prior to the use of the stoma guard 100.

The stoma guard 100 offers those who have undergone an ostomy procedure a simple and sure means of precisely securing an ostomy stoma 10 and enables ostomy wearers to eliminate the risk of blockage. With a simple snap, the stoma guard 100 is quickly and easily secured. Further, use of the stoma guard 100 can effectively prevent premature leaks which so often occur when the stoma 10 is blocked. Consequently, ostomy wearers may be spared the embarrassment and discomfort associated with a leaking pouch, as well as the skin irritation and infection which can result. The stoma guard 100 may be produced in a universal size or in various sizes to accommodate various needs and thus may be utilized by any child, teen, or adult who has undergone a urostomy, ileostomy or colostomy procedure. The stoma guard 100 fits both the one and two piece systems and is disposable. Additionally, the stoma guard 100 is an invaluable accessory in home health care, hospitals, nursing homes, and any other medical establishment.

The description of the present invention has been presented for purposes of illustration and description, but is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated.

What is claimed is:

1. A stoma protective device, comprising:
a circular base ring having a top surface;
a cylindrical wall extending from the top surface of the base ring and having a diameter less than a diameter of the base ring; and
a hood extending from the cylindrical wall, the hood having a funnel shape with an opening through a top portion extending through at least a portion of a side portion;
the base ring configured to be secured between a flange secured to a patient's stoma and a ring secured to an ostomy pouch, whereby the hood protrudes into the interior of the ostomy pouch.

2. The stoma protective device, wherein the base ring, wall, and hood are formed from a disposable material.

3. The stoma protective device of claim 1, wherein the base ring, wall, and hood are formed as single integrated piece.

4. The stoma protective device of claim 1, wherein:
the upper surface of the base ring has a diameter from about 0.25 inches to about 0.5 inches;
the wall has a diameter from about 0.25 inches to about 0.5 inches;
the base ring has a height from about 0.002 inches to about 0.003 inches;
the wall has a height from about 0.04 inches to about 0.06 inches above the upper surface of the base ring; and
the hood has a height from about 0.01 inches to about 0.02 inches above the wall.

5. The stoma protective device of claim 1, wherein:
the upper surface of the base ring has a diameter of about 0.4716 inches;
the wall has a diameter of about 0.4356 inches;
the base ring has a height of about 0.035 inches;
the wall has a height of about 0.0503 inches above the upper surface of the base ring; and
the hood has a height of about 0.013 inches above the wall.

6. The stoma protective device of claim 1, wherein a lower surface of the base ring has a diameter less than the diameter of the upper surface.
